(19) United States
(12) Patent Application Publication
(10) Pub. No.: US 2025/0160810 A1
(43) Pub. Date: May 22, 2025

(54) Modular Hip Distractor

(71) Applicant: The University of Toledo, Toledo, OH (US)

(72) Inventors: Sophia M. Soehnlen, Toledo, OH (US); Italia Fernandez, Toledo, OH (US); Benjamin T. Bisesi, Toledo, OH (US); Avik N. Mukherjee, Toledo, OH (US); Yogesh Kumaran, Toledo, OH (US); David Sohn, Toledo, OH (US)

(73) Assignee: The University of Toledo, Toledo, OH (US)

(21) Appl. No.: 18/951,851

(22) Filed: Nov. 19, 2024

Related U.S. Application Data
(60) Provisional application No. 63/600,914, filed on Nov. 20, 2023.

(51) Int. Cl. A61B 17/02; A61H 1/02

(52) U.S. Cl. CPC A61B 17/025; A61B 17/0218; A61H 1/0255; A61B 2017/0275; A61H 2201/0103; A61H 2201/1436; A61H 2201/164; A61H 2201/165; A61H 2205/088

(57) ABSTRACT

A modular hip distractor and methods of using the same are described. The modular hip distractor includes a knee brace assembly having at least one cuff and that is configured to reduce the amount of force applied to the knee joint and lower extremity during a hip distraction procedure.

### RELATED APPLICATIONS

This application claims priority to U.S. Provisional Application No. 63/600,914 filed under 35 U.S.C. § 111(b) on Nov. 20, 2023, the disclosure of which is incorporated herein by reference in its entirety for all purposes.

### STATEMENT REGARDING FEDERALLY SPONSORED RESEARCH

This invention was made with no government support. The government has no rights in the invention.

### BACKGROUND

Hip distraction is a minimally invasive procedure where surgical tools are inserted into the patient's hip via small incisions. The procedure is utilized in the treatment of a wide range of hip problems, such as femoroacetabular impingement (FAI), labral tears, and the removal of loose bodies. During hip distraction, the hip joint needs to be moved a sufficient space to provide an ample view of the surgical area for the surgeon. Ideally, the leg on the same side (ipsilateral) as the hip joint being operated on will undergo traction, resulting in distraction. Traction is typically performed by first attaching boots to secure the patient's legs, and most commonly, placing a perineal post against the patient's groin to serve as an anchor point during traction. Then, the ipsilateral leg is pulled using a traction system, thus temporarily separating the femoral head from the acetabulum. However, this procedure poses an increased risk of adverse outcomes in patients with preexisting joint pathologies, especially those with prior total knee replacements (TKRs). In fact, many patients undergoing hip distractions experience traction-related problems, with complaints commonly related to the knee joint. Thus, as the rate of hip distraction procedures continually rises, there is a need to protect patients with compromised joints during surgery from enduring additional complications.

### SUMMARY

Provided herein is a modular hip distractor comprising a first leg support member extending from a first proximal end at a junction to a first distal end; a second leg support member extending from a second proximal end at the junction to a second distal end; a first foot brace on the first leg support member; a first knee brace assembly on the first leg support member between the first proximal end and the first foot brace; and a crank system configured to move the first foot brace and the first knee brace in concert along the first leg support member; wherein the junction is configured to extend from, or otherwise attach to, an operating bed.

In certain embodiments, the first knee brace assembly comprises a first cuff and a second cuff, wherein the first cuff is disposed along the first leg support member between the junction and the second cuff. In particular embodiments, the first cuff has a first diameter, the second cuff has a second diameter, and the first diameter is larger than the second diameter. In particular embodiments, the first cuff and the second cuff define openings substantially axially parallel with a longitudinal axis of the first leg support member. In particular embodiments, each of the first cuff and the second cuff are mesh-like structures. In particular embodiments, the first cuff is attached to a first cuff support comprising a plurality of arms, the mesh-like structure of the first cuff being adhered to the plurality of arms of the first cuff support; and the second cuff is attached to a second cuff support comprising a plurality of arms, the mesh-like structure of the second cuff being adhered to the plurality of arms of the second cuff support. In particular embodiments, the first cuff support is connected to the second cuff support through a pivot configured to allow relative movement of the first cuff support and the second cuff support. In particular embodiments, the first cuff has a first inflator configured to inflate the first cuff, and/or the second cuff has a second inflator configured to inflate the second cuff. In particular embodiments, the modular hip distractor further comprises a first pressure gauge configured to measure a pressure exerted on a lower extremity of a patient.

In certain embodiments, the first knee brace assembly is movable longitudinally along a track in the first leg support member, and the first foot brace is movable longitudinally along the track. In particular embodiments, the first knee brace assembly and the first foot brace are configured to move upon movement of a belt within the first leg support member.

In certain embodiments, the modular hip distractor further comprises a crank and belt, the belt extending within the first leg support member in an infinite loop between a first pulley near the distal end and a second pulley near the proximal end, wherein the belt is movable in the infinite loop by rotation of the crank. In particular embodiments, the modular hip distractor further comprises a knee brace base connected to a knee brace sliding member disposed on a track on a surface of the first leg support member, wherein the knee brace sliding member is connected to the belt; and a foot brace base connected to a foot brace sliding member disposed on the track and connected to the belt; wherein the first knee brace assembly is connected to the knee brace base, and the first foot brace is connected to the foot brace base; and wherein the knee brace base and the foot brace base are configured to move in concert upon movement of the belt in the infinite loop. In particular embodiments, the knee brace base includes a first tab that fits within a channel of the knee brace sliding member, and the foot brace base includes a second tab that fits within a channel of the foot brace sliding member. In particular embodiments, the knee brace base is attached to a knee brace connector piece so as to surround or encircle the first leg support member, and the foot brace base is attached to a foot brace connector piece so as to surround or encircle the first leg support member. In particular embodiments, the first foot brace is attached to the foot brace base through a foot brace support structure, and the knee brace base is attached to the knee brace through a knee brace support structure. In particular embodiments, the knee brace support structure comprises a first branch support connected to a second branch support through a pivot, wherein the first branch support comprises a plurality of curved arms attached to a first cuff of the knee brace assembly, and wherein the second branch support comprises a plurality of curved arms attached to a second cuff of the knee brace assembly, wherein the first cuff and the second cuff each comprise a mesh-like material.

In certain embodiments, the crank system comprises a crank, a first pulley, a second pulley, and a belt running in an infinite loop between the first pulley and the second pulley, wherein the crank is operable to move the belt along the infinite loop.

In certain embodiments, the modular hip distractor further comprises a second foot brace on the second leg support member.

In certain embodiments, the modular hip distractor further comprises a second knee brace assembly on the second leg support member.

Further provided is a modular hip distractor comprising first and second leg support members configured to support first and second legs of a patient disposed on an operating bed, wherein at least one of the first and second leg support members includes a knee brace assembly configured to reduce a force exerted on a knee of the first leg of the patient while a hip of the first leg of the patient is distracted in the modular hip distractor.

Further provided is a modular hip distractor comprising leg support members configured to support legs of a patient disposed in a supine or Trendelenburg position while a hip of a first leg of the patient is distracted, wherein one of the leg support members includes a knee brace assembly configured to receive a knee of the first leg of the patient and reduce an amount of force exerted on the knee while the hip is distracted.

Further provided is a method of performing a hip distraction, the method comprising configuring a patient in a supine, Trendelenburg, lateral decubitus, or prone position; securing a knee joint of a first leg of the patient with a knee brace assembly, wherein the knee brace assembly comprises a first cuff configured to receive a thigh region of the first leg and a second cuff configured to receive a calf region of the first leg; securing a foot of the first leg in a foot brace; and pulling traction on the first leg by pulling a sufficient force simultaneously to the knee brace assembly and the foot brace to cause separation of a femoral head of the first leg from an acetabulum of the first leg.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Perspective view of a non-limiting example embodiment of a modular hip distractor.

FIG. 2: Perspective view of the modular hip distractor shown without a foot brace.

FIG. 3: Close-up view of the modular hip distractor showing a knee brace assembly.

FIG. 4: Top view of the modular hip distractor.

FIG. 5: Perspective view of the modular hip distractor with a foot brace on each of the first and second leg support members.

FIG. 6: Graph showing hip distraction versus stabilization force in a test described in the examples herein.

FIGS. 7A-7B: Illustrations depicting a patient in a lateral decubitus (FIG. 7A) or prone (FIG. 7B) position for a hip distraction procedure.

### DETAILED DESCRIPTION

Throughout this disclosure, various publications, patents, and published patent specifications may be referenced by an identifying citation. The disclosures of these publications, patents, and published patent specifications are hereby incorporated by reference into the present disclosure in their entirety to more fully describe the state of the art to which this invention pertains.

Provided herein is a modular hip distractor with a knee brace to be used during hip distraction procedures to reduce the force applied to the knee joint during traction. The knee brace can be positioned around a patient's lower thigh, entire knee, and/or upper calf, and can result in traction being achieved at a better rate and in a safer manner. A mesh-like structure can come into contact with the patient's skin to prevent slippage of the knee brace as traction is applied to the patient's leg. Typically during traction the patient's foot and ankle are secured in a boot using straps (and possibly by wrapping multiple layers of gauze around the boot). However, this method of restraint is not necessary to be replicated on the knee and its surrounding structures with the knee brace described herein. The knee brace, which can be in the form of an assembly divided into upper/thigh and lower/calf portions, can be attached to the same leg support member that runs longitudinally with the patient's leg and connects to the boot holding the patient's ipsilateral foot. The knee brace is part of a modular system with components that can be added and removed easily for the desired application. Whereas conventional hip distractor systems seek to control a range of motion of the affected limb, the modular hip distractor described herein is configured to protect a patient's knee during hip distraction surgery.

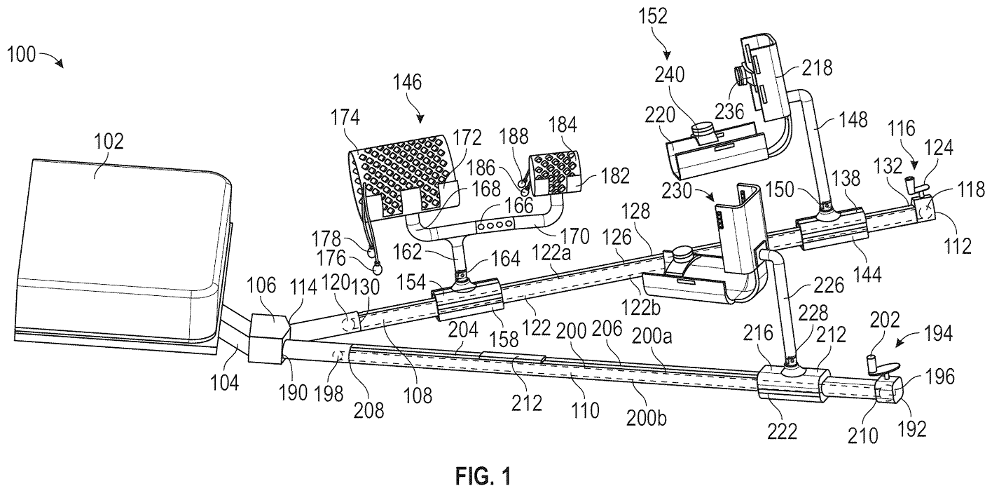

Referring now to FIGS. 1-4, a non-limiting example embodiment of a modular hip distractor 100 is depicted. The modular hip distractor 100 can be attached to an operating bed 102. However, the modular hip distractor 100 may alternatively include a bed 102 integrated with the modular hip distractor 100. It is understood that the operating bed 102 can be any surface on which a patient undergoing a hip distraction procedure is disposed. The modular hip distractor 100 is compatible with surgical beds and may also be attached to some conventional hip distraction systems. In embodiments in which the modular hip distractor 100 is attached to an operating bed 102, the modular hip distractor 100 includes a junction 106 connected to the operating bed 102 through a support bar 104. In alternative embodiments in which the modular hip distractor 100 does not connect to an operating bed, or connects to a conventional hip distraction system, the modular hip distractor 100 may not include the support bar 104.

Referring still to FIGS. 1-4, the modular hip distractor 100 includes a first leg support member 108 and a second leg support member 110 each extending from the junction 106 in a direction away from the operating bed 102. In other words, the first leg support member 104 and the second leg support member 110 extend from the junction 106 on the side opposite the support bar 104 (when the support bar 104 is present). The first leg support member 108 and the second leg support member 110 are configured to independently translate vertically and/or horizontally due to a hinge joint, or any other suitable configuration, within the junction 106. Further, the first leg support member 108 and/or the second leg support member 110 are configured to be able to be removed from the modular hip distractor 100 if either is not needed during the procedure. The first leg support member 108 and second leg support member 110 can be identical in that they may contain the same components; however, the first leg support member 108 and the second leg support member 110 need not be identical. The first and second leg support members 108, 110 should be rigid enough to support the weight of any components attached to them and to retain the ability to remain untethered to the ground (except through the operating bed 102 or other structure opposite the junction 106 from the first and second leg support members 108, 110) so that they may be freely moved by a surgeon performing a hip distraction procedure. The first and second leg support members 108, 110 function to receive, support, and immobilize a patient's first and second legs, respectively, during a hip distraction procedure. Each of the first and second leg support members 108, 110 can extend, for example, from a position underneath a patient's mid or lower thigh to a position underneath the patient's foot when the patient is in position on the operating bed 102 with their legs mounted on the first and second leg support members 108, 110.

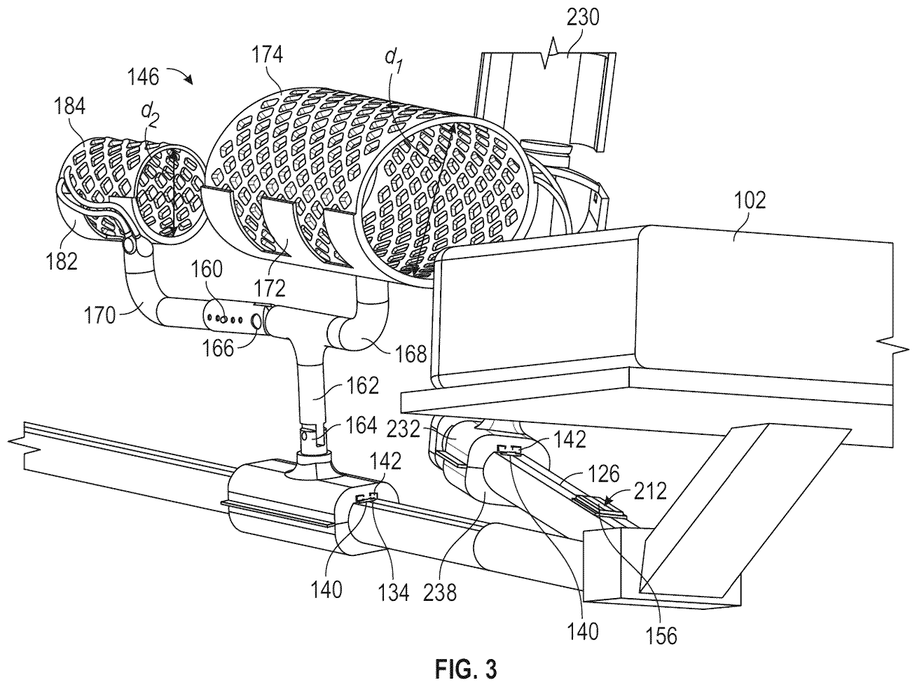

Referring still to FIGS. 1-4, the first leg support member 108 extends from a proximal end 114 (at the junction 106) to a distal end 112. As best seen in FIG. 3, each of the first and second leg support members 108, 110 includes a track 126, which is a longitudinal slit providing access to an internal area within the respective leg support member 108, 110. The first leg support member 108 includes a first crank system 116 which includes a first belt 122 running in an infinite loop within the first leg support member 108, where the infinite loop includes a top portion 122a and a bottom portion 122b and engages a first pulley 118 and a second pulley 120. The first pulley 118 is at the distal end 112 of the first leg support member 108, and the second pulley 120 is at the proximal end 114 of the first leg support member 108. However, the pulleys 118, 120 do not need to be pulleys. Rather, the pulleys 118, 120 can be merely standard wheels, optionally with teeth for enhanced gripping of the first belt 122.

The first crank system 116 includes a first crank 124 disposed at the distal end 112 of the first leg support member 108, where the first crank 124 can include a ratchet or locking mechanism so as to prevent unwanted movement of the first crank 124 in either direction. Rotation of the first crank 124 causes rotation of the first pulley 118 which, in turn, causes movement of the first belt 122 in the infinite loop. The first belt 122 may include teeth that are configured to be received by grooves in the first pulley 118. As such, when the first pulley 118 rotates from the action of the first crank 124, the first belt 122 also moves. In another non-limiting example, the tension on the first belt 122 between the first pulley 118 and the second pulley 120 is sufficient to cause movement of the first belt 122 upon rotation of the first pulley 118 by the first crank 124. However, other crank and belt systems are possible and encompassed within the scope of the present disclosure.

As noted above, the first pulley 118 is rotatable by action of the first crank 124 disposed at the distal end 112 of the first leg support member 108. The second pulley 120 is in mechanical communication with a first crank 124 via the first belt 122. As the first crank 124 is turned (e.g., by the surgeon performing the hip distraction procedure), the first pulley 118 rotates which, in turn, causes the first belt 122 to move along its infinite loop between a top portion 122a and a bottom portion 122b. The first belt 122 moves in a first direction in the top portion 122a and moves in an opposite second direction in the bottom portion 122b. In the top portion 122a, the first belt 122 may be grasped by, or otherwise connected to, various sliding members disposed on the track 126. This provides the ability for the surgeon to manually move first belt 122 along the loop which, as will be described in more detail, can move components along the first track 126 to accomplish traction while supporting the knee joint of the patient.

Referring to FIG. 1 and FIG. 3, the track 126 extends on a first surface 128 of the first leg support member 108 from a proximal point 130 to a distal point 132. The proximal point 130 is near, but does not extend to, the second pulley 120, and the distal point 130 is near, but does not extend to, the first pulley 118. The first track 126 is an area of the first leg support member 108 where components attached to the first belt 122 may traverse a longitudinal axis between the proximal end 114 and the distal end 112 of the first leg support member 108 by riding along the first belt 122. However, the track 126 is not so large of a slit in the first surface 128 so as to allow any such components to rest their weight on the first belt 122 instead of the rigid material of the first leg support member 108. Thus, the track 126 functions to provide access to movement with the first belt 122 without impeding the movement of the first belt 122 by imposing weight on the first belt 122.

Referring still to FIGS. 1-4, one such component which may traverse the track 126 is a knee brace assembly 146. Another such component which may traverse the track 126 is a foot brace 152. Leg rotation is common during surgery. Therefore, the first track 126 allows for the knee brace assembly 146 to be moved in concert with the foot brace 152, while preserving the ability for the knee brace assembly 146 and the foot brace 152 to move independently (by simply removing one of the knee brace assembly 146 or foot brace 152 while moving the other along the track 126), as described in more detail below. As noted above and as demonstrated in the examples herein, the inclusion of a knee brace assembly 146 allows for reduced forces to be exerted on the knee joint during traction.

Disposed on the track 126 is a first knee brace sliding member 134 and a first foot brace sliding member 136. Each of the first knee brace sliding member 134 and the first foot brace sliding member 136 is attached to the top portion 122a of the first belt 122. The first knee brace sliding member 134 is configured to attach the knee brace assembly 146 to, and support the knee brace assembly 146 on, the first leg support member 108 while allowing for movement of the knee brace assembly 146 along the track 126. The first foot brace sliding member 136 is configured to attach the foot brace 152 to, and support the foot brace 152 on, the first leg support member 108 while allowing for movement of the foot brace 152 along the track 126. The first foot brace sliding member 136 is distal to the first knee brace sliding member 134. In other words, the first knee brace sliding member 134 is disposed on the first leg support member 108 between the junction 106 and the first foot brace sliding member 136, and the first foot brace sliding member 136 is disposed between the first knee brace sliding member 134 and the distal end 112. The first crank system 116 is configured to move the first knee brace sliding member 134 and the first foot brace sliding member 136 in concert along the track 126. As the first crank 124 is rotated, the first foot brace sliding member 136 and the first knee brace sliding member 134 move together in the same direction and at the same rate. However, although the first crank system 116 is illustrated and described in this way, any desired mechanism or method for moving the first knee brace sliding member 134 and the first foot brace sliding member 136 along the track 126 may be utilized.

Referring to FIGS. 1 and 3, the modular hip distractor 100 includes an adjustable and removable knee brace assembly 146. The knee brace assembly 146 is a structural brace system that creates additional support around the patient's knee joint by providing a knee brace situated directly above and below the knee. The knee brace assembly 146 includes a first cuff 174 and a second cuff 184 connected by a first branch support 168 and a second branch support 170, and connects to the first leg support member 108 with a knee brace base 154 which attaches to the first knee brace sliding member 134. Each of the first cuff 174 and the second scuff 184 defines an opening that is substantially parallel with a longitudinal axis of the first leg support member 108, although this can be changed by the pivots 164, 166. The first branch support 168 and the second branch support 170 are jointed together by a pivot 166 which allows the first and second branch supports 168, 170 to pivot relative to one another. This allows for patient legs of different shapes and sizes, as well as different positions, to be accommodated easily. The pivot 166 can further have an extension feature 234 which may include a locking mechanism and an extension mechanism to extend or retract the distance between the first cuff 174 and the second cuff 178.

Referring to FIG. 1 and FIG. 3, the knee brace base 154 is movable longitudinally along the track 126 by action of the first crank system 116 as discussed above. Specifically, the knee brace base 154 is attachable to the first knee brace sliding member 134. The first knee brace sliding member 134 is a plate or other structure configured to slide along the track 126. As best seen in FIG. 3 where a second knee brace sliding member 212 (identical in structure to the first knee brace sliding member 134 on the first leg support member 108) is illustrated on the second leg support member 110 without any components on it, the knee brace sliding member 134 includes a channel 156 and also includes a protrusion extending into the track 126 configured to attach to the first belt 122 in the top portion 122a. The first knee brace sliding member 134 can attach to the first belt 122 with any suitable fastener such as, but not limited to, staples, screws, adhesive, or a combination thereof. The first knee brace sliding member 134 provides a point of attachment for the knee brace assembly 146 to the first leg support member 108 with structural integrity and an ability to slide along the track 126. As the first crank system 116 is actuated, and the first belt 122 is moved along the infinite loop in the track 126, the first knee brace sliding member 134 slides with the top portion 122a of the first belt 122.

Referring to FIGS. 1-4, the knee brace base 154 is attached to the first knee brace sliding member 134 on the first leg support member 108. As can be seen best in FIG. 3, the knee brace base 154 has a portion 140 that fits within the channel 156 of the first knee brace sliding member 134 so as to connect the knee brace base 154 to the first knee brace sliding member 134. To remove the knee brace base 154 from the first knee brace sliding member 134, the knee brace base 154 may be slid out of the channel 156 while holding the knee brace sliding member 134 still. A first knee brace connector piece 158 attaches to the knee brace base 154 so as to form a structure surrounding or encircling the first leg support member 108 while still able to traverse the track 126 on the first leg support member 108. The first knee brace connector piece 158 can be attached to the knee brace base 154 with any suitable fasteners such as screws. However, other methods of connecting the knee brace base 154 and the first knee brace connector piece 158 are possible and encompassed within the scope of the present disclosure. Other non-limiting example means of connecting these pieces include a snap fit connection, a nut and bolt connection, a magnetic connection, or the like.

Referring to FIG. 1 and FIG. 3, extending from the knee brace base 154 is a support structure 162 having pivots 164, 166. The support structure 162 branches off to form the first branch support 168 and the second branch support 170, which are joined by the pivot 166. At the distal end of the first branch support 168 is a first cuff support 172 which secures the first cuff 174 in place. The first cuff support 172 may be formed from a plurality of curved arms to which the first cuff 174 attaches via a suitable adhesive. The first cuff 174 can be one-piece, multiple pieces, one piece attached by hook-and-loop fasteners, or the like. It is advantageous for the first cuff 174 to be one piece as it leaves less room for errors during a procedure. The first cuff 174 has a diameter d₁, and is configured to house, restrain, hold, grasp, encircle, or otherwise touch an area of a patient's leg slightly proximal to the knee joint (i.e., a thigh region of the patient's leg). The first cuff 174 has a first inflator 176 and a first pressure gauge 178. The first cuff 174 can be inflated with the first inflator 176, and the first pressure gauge 178 can be used to monitor how much pressure is being applied to that portion of a patient's knee.

The first cuff 174 is inflated via the first inflator 176. The first cuff 174 and first inflator 176 work as a mechanism of tightening a brace around the patient's leg which is advantageous to other techniques such as the use of straps. Using a cuff and inflator allows one to monitor the pressure exerted on the patient's leg (via the first pressure gauge 178) to standardize the minimum required force needed for the first cuff 174 to be securely attached on the leg while traction is being pulled. This is advantageous because it allows for the force (i.e., the compressive force of the first cuff 174 surround the leg) to be evenly distributed throughout the entire first cuff 174, which is a larger area than if straps were used instead. A strap would provide high compressive forces on the leg that are concentrated to the small area of the leg that the strap covers, creating opportunity to injure the underlying anatomical structures. The use of a cuff and inflator as described herein is a solution to this problem, because the resulting compressive forces are spread over a larger area. However, other mechanisms which fix the knee rigidly are possible and encompassed within the scope of the present disclosure.

The first pressure gauge 178 is configured to measure and display or communicate the pressure exerted on the patient's leg by the first cuff 174 (i.e., stemming from the degree to which the first cuff 174 is inflated). The first pressure gauge 178 may generate a warning when the pressure or time may reach dangerous levels. This can be used in addition to current methods such as x-ray imaging to determine the risk of injury to surrounding anatomical structures during hip distraction procedures.

Referring still to FIG. 1 and FIG. 3, the first cuff 174 is proximal to the second cuff 184. At the proximal end of the second branch support 170 is a second cuff support 182 which secures the second cuff 184 in place. The second cuff support 182 may be formed from a plurality of curved arms to which the second cuff 184 attaches via a suitable adhesive. The second cuff 184 has a diameter d₂ which is smaller than the diameter d₁ of the first cuff 174. The second cuff 184 is configured to house, restrain, hold, grasp, encircle, or otherwise touch an area of a patient's leg slightly distal to the knee joint (i.e., a calf region of the patient's leg). The second cuff 184 can be one-piece, multiple pieces, one piece attached by hook-and-loop fasteners, or the like. It is advantageous for the second cuff 184 to be one piece as it leaves less room for errors during a procedure. The second cuff 184 has a second inflator 186 and a second pressure gauge 188. The second cuff 184 can be inflated with the second inflator 186, and the second pressure gauge 188 can be used to determine how much pressure is being exerted on that portion of a patient's knee by the second cuff 184 in the same manner as the first pressure gauge 178 with respect to the first cuff 174 as described above. The second pressure gauge 188 can measure and display or communicate the force applied to the relevant portion of the patient's knee.

Referring still to FIG. 1 and FIG. 3, the first cuff 174 and the second cuff 184 each have a mesh-like structure which makes contact with the patient's skin to enhance the surface area and prevent slippage of the knee brace assembly 146 as traction is applied to the patient's leg, further reducing the potential adverse events of over-compression of anatomical structures. The first and second cuffs 174, 184 are typically non-cloth-based so as to allow for proper cleansing of parts near the operative site and reduce potential adverse events of infection. The first and second cuffs 174, 184 can be composed of a polymer mesh material that offers advantages intraoperatively for the surgeon and patient. Unlike conventional compression strapping, mesh materials allow for a larger surface area to be in contact with the skin while not constricting the leg over the full surface area. The polymer-based material allows for a greater frictional coefficient compared to other materials so as to minimize slippage during surgery. The first and second cuffs 174, 184 are configured to have multiple contact points around the patient's knee, and using a polymer-based material allows for proper sterility needed since the knee brace assembly 146 is close to the surgical sites. While it is not necessary that the first cuff 174 and the second cuff 184 each be mesh materials, it is not desirable to use narrow straps to form the first cuff 174 and the second cuff 184. To lower the risk of neurovascular complications, a wider mesh is better to help prevent the knee brace assembly 146 from slipping during traction while at the same time avoiding creating pressure points on the patient's leg. Avoiding the use of narrow straps prevents constriction of the patient's lower limbs. The cuffs 174, 184 can accommodate legs of different circumference due to the nature of the inflation mechanism.

In use, the knee brace assembly 146 can be moved along the track 126 by action of the first crank system 116. The knee brace assembly 146 can be moved in this manner in concert with a foot brace 152 configured to securely hold the patient's foot of the same leg and provide an additional anchoring point during traction. In other words, both the knee brace assembly 146 and the foot brace 152 can apply traction simultaneously so as to reduce the traction force which would otherwise be required if no knee brace were present.

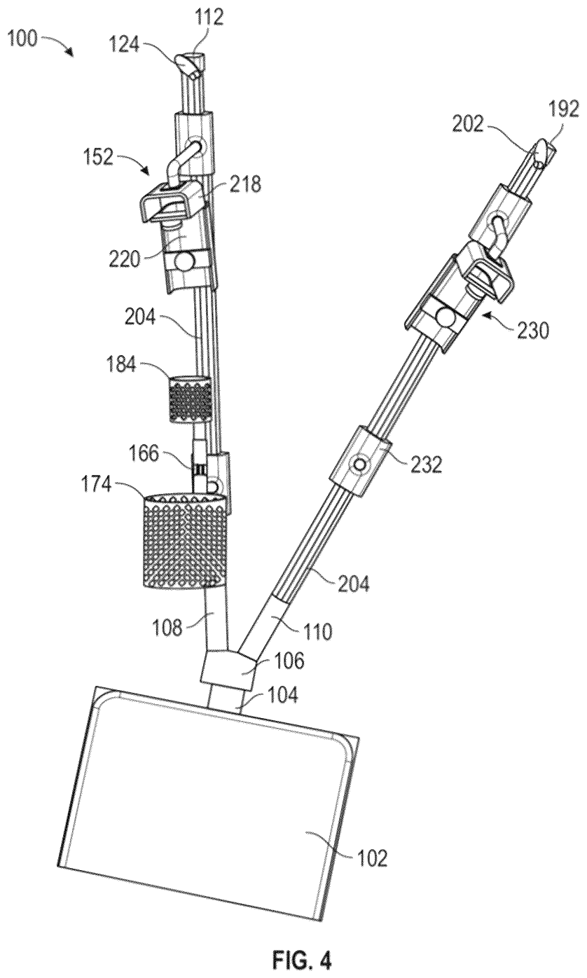

Referring now to FIG. 1 and FIG. 4, the modular hip distractor includes the foot brace 152 at the distal end 112 of the first leg support member 108. The foot brace 152 is mounted on the first leg support member 108 and is movable along the track 126 in the same manner as the knee brace assembly 146. That is, the foot brace 152 is connected to a foot brace sliding member 136 disposed on the track 126 and connected to the top portion 122a of the first belt 122 so as to move along the track 126 with movement of the first belt 122. The foot brace sliding member 136 is identical in structure to the knee brace sliding member 134 discussed above. That is, the foot brace sliding member 136 is a plate or other structure configured to slide along the track 126, and includes a channel and a protrusion extending into the track 126 configured to attach to the first belt 122 in the top portion 122a. Because both the foot brace sliding member 136 and the first knee brace sliding member 134 are attached to the first belt 122 in the top portion 122a, they move in the same direction as each other (i.e., at the same rate) with movement of the first belt 122. The foot brace sliding member 136 can attach to the first belt 122 with any suitable fastener such as, but not limited to, staples, screws, adhesive, or a combination thereof. The foot brace sliding member 136 provides a point of attachment for the foot brace 152 to the first leg support member 108 with structural integrity and an ability to slide along the track 126.

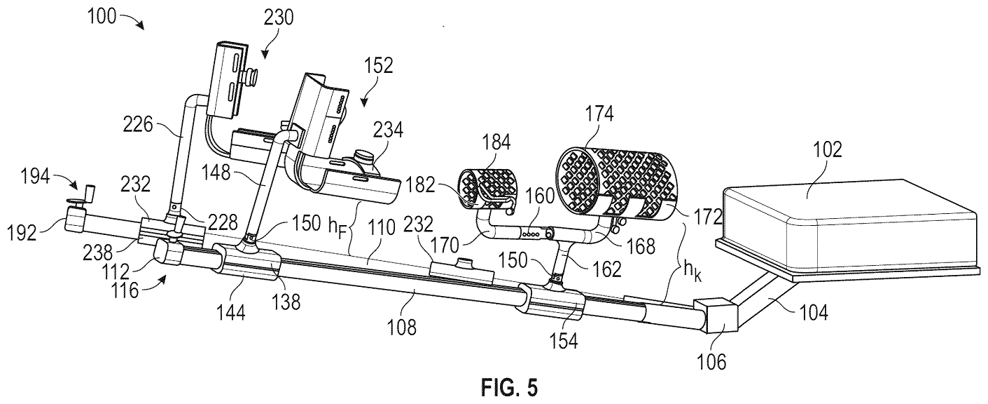

Referring to FIG. 1 and FIG. 5, the modular hip distractor 100 includes a foot brace base 138 which is removably connected to the foot brace sliding member 136 in the same manner, and utilizing identical structural components, as the knee brace base 154 is removably connected to the knee brace sliding member 134. That is, the foot brace base 138 includes a portion or tab which slides into a channel on the foot brace sliding member 136. Referring to FIG. 5, a foot brace connector piece 144 attaches to the foot brace base 138 in the same manner, and utilizing identical structural components, as the first knee brace connector piece 158 attaches to the knee brace base 154. That is, the foot brace connector piece 144 and the foot brace base 138 form a structure surrounding or encircling the first leg support member 108 while still able to traverse the track 126 on the first leg support member 108 via movement of the foot brace sliding member 136. The foot brace connector piece 144 can be attached to the foot brace base 138 with any suitable fasteners such as screws. However, other methods of connecting the foot brace connector piece 144 to the foot brace base 138 are possible and encompassed within the scope of the present disclosure. Other non-limiting example means of connecting these pieces include a snap fit connection, a nut and bolt connection, a magnetic connection, or the like.

Referring to FIG. 1 and FIG. 5, the foot brace 152 is attached to the foot brace base 138 through the support structure 148. The foot brace 152 is configured to securely hold the foot of the same leg of the patient that is being distracted for the hip distraction procedure. The foot brace 152 includes a plantar support 218 and an achilles support 220 which may be disposed in a substantially orthogonal manner relative to each other. Each of the plantar support 218 and the achilles support 220 may include one or more straps 236, 240 to help secure the patient's foot in a manner which can prevent rotation or other unwanted movement. The plantar support 218 may include a dorsum strap 236, and the achilles support 220 may include an ankle strap 240.

Referring to FIG. 1 and FIG. 5, the support structure 148 is an elongate member extending above the first leg support member 108 in a manner so as to dispose the foot brace 152 at a height h_{F} above the first leg support member 108 roughly close to the height h_{K} the knee brace assembly 146 is disposed above the first leg support member 108. In other words, the foot brace 152 and the first and second cuffs 174, 184 of the knee brace assembly 146 are roughly aligned so as to receive and secure a leg of a patient disposed on the operating bed 102. The support structure 148 may include a pivot 150 configured to allow the foot brace 152 to pivot relative to the first leg support member 108 so as to accommodate different leg positions, sizes, and adjustments.

The first foot brace base 138 is removable as desired, such as if the corresponding leg of a patient is not to be distracted. The first foot brace base 138 can be removed from the first track 126 simply by removing the fasteners connecting the foot brace base 138 to the foot brace connector piece 144, and sliding the foot brace base 138 out of the channel of the foot brace sliding member 136 while holding the foot brace sliding member 136 still.

Referring to FIG. 1, upon rotation of the first crank 116, the first belt 122 moves around the first and second pulleys 118, 120 in the top portion 122a and bottom portion 122b, and carries with it the attached foot brace base 138 (attached to the first belt 122 via the foot brace sliding member 136) and the attached knee brace base 154 (attached to the first belt 122 via the knee brace sliding member 134). In this manner, both the knee brace assembly 146 and the foot brace 152 move in concert either distally or proximally along the longitudinal axis of the first leg support member 108, so as to apply or release traction to the patient's ipsilateral leg, respectively. This allows for the hip of the patient to be distracted with overall reduced traction forces, therefore reduced forces being exerted on the patient's knee joint compared to conventional hip distraction methods.

A relatively large traction force can be applied to separate the femoral head from the acetabulum (a step performed during hip distraction procedures). However, because the knee brace assembly 146 and the foot brace 152 are both connected to the first belt 122 and configured to traverse the track 126 in concert upon movement of the first belt 122 through actuation of the first crank system 116, the presence of the knee brace assembly 146 effectively reduces the force needed to achieve hip distraction and reduces the force exerted on the knee joint during hip distraction. When traction is applied, instead of merely applying a pulling force on the patient's foot, the force is distributed simultaneously to the patient's foot and to multiple areas around the patient's lower extremity, so long as the first cuff 174 and the second cuff 184 are securely attached to the patient's leg. In this manner, the knee brace assembly 146 is capable of lowering the total traction force during a hip distraction procedure, and the force exerted on the knee joint. As demonstrated in the examples herein, testing has shown that a knee brace has the ability to lower the overall traction force used in a hip distraction. As such, the modular hip distractor 100 can reduce the number of patients who experience traction related complications, such as knee pain or joint instability following a hip distraction procedure.

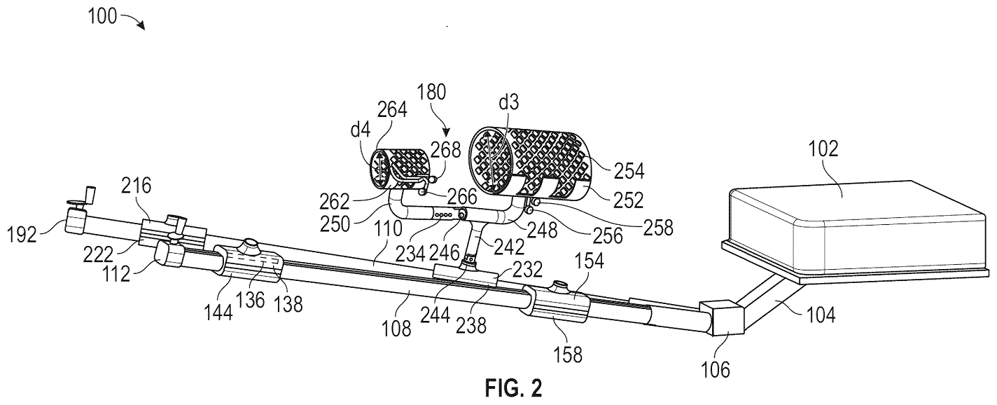

All of the above components mounted on or otherwise connected to the first leg support member 108, for traction of a patient's first leg, can be duplicated or alternatively present on the second leg support member 110 to facilitate traction of the patient's second leg. That is, the modular hip distractor 100 may include two knee brace assemblies (one for each of the patient's knees), and two foot braces (one for each of the patient's feet), or may include only one knee brace assembly and only one foot brace. Other combinations are also possible. For instance, the modular hip distractor 100 may include two foot braces (one on each leg support member 108, 110) but only one knee brace assembly. Similarly, the modular hip distractor 100 may include two knee brace assemblies (one on each leg support member 108, 110) but only one foot brace. Also, the modular hip distractor 100 may include sliding members or brace and connector pieces in the absence of the structures they support, such as depicted in FIG. 2, where the modular hip distractor 100 is illustrated with two foot brace bases 138, 232 with respective connector pieces 144, 222 on sliding members but no foot brace, one knee brace assembly 180, and one knee brace base and connector piece on a sliding member without a second knee brace assembly. Thus, the modular hip distractor 100 is versatile and adaptable.

Referring to FIGS. 1-4, components of the second leg support member 110 may be identical in structure to the components described above with respect to the first leg support member 108. The second leg support member 110 extends from a proximal end 190 to a distal end 192, and includes a second crank system 194 which functions in the same manner as the first crank system 116. The second crank system 194 includes a third pulley 196 and a fourth pulley 198 that are in communication with a second belt 200 disposed within the second leg support member 110, and running in an infinite loop that includes a top portion 200a and a bottom portion 200b. The second belt 200 engages the third pulley 196 and the fourth pulley 198. The second belt 200 may include teeth configured to be received by grooves in the third pulley 196 such that the second belt 200 moves with rotation of the third pulley 196. Alternatively, the tension on the second belt 200 between the third pulley 196 and the fourth pulley 198 may be sufficient to force movement of the second belt 200 upon rotation of the third pulley 196. The third pulley 196 rotates upon rotation of the second crank 202. The second crank 202 can include a ratchet or locking mechanism so as to prevent unwanted movement in either direction. However, other pulley and belt systems are possible and encompassed within the scope of the present disclosure.

Referring to FIG. 1 and FIG. 4, the second leg support member 110 includes a track 204 that extends on a first surface 206 from a proximal point 208 to a distal point 210. Disposed on the second track 204 is a second knee brace sliding member 212 and a second foot brace sliding member 214. Each of the second knee brace sliding member 212 and the second foot brace sliding member 214 is attached to the top portion 200a of the second belt 200. The second crank system 194 is configured to move the second knee brace sliding member 212 and the second foot brace sliding member 214 in concert along the second track 204. As the second crank 202 is rotated, the sliding members 212, 214 move together in the same direction and at the same rate based on their mutual connection to the top portion 200a of the second belt 200. However, other ways of moving the second knee brace sliding member 212 and the second foot brace sliding member 214 may be utilized. For example, the second crank system 194 can be configured to move the sliding members 212, 214 independently of each other.

Referring to FIGS. 1-4, a second foot brace base 216 can be attached to a second foot brace sliding member 214 in the same manner that the first foot brace base 138 is attached to the first foot brace sliding member 136. The second foot brace base 216 has a portion or tab which fits within a channel of the second foot brace sliding member 214. A second foot brace connector piece 222 connects to the second foot brace base 216 with suitable fasteners, such as screws, so as to form a structure which encircles or surrounds the second leg support member 110 while still being capable of sliding along the second track 204. Other acceptable methods of connecting the second foot brace connector piece 222 to the second foot brace base 216 can include, but are not limited to, a snap fit connection, a nut and bolt connection, or a magnetic connection.

Referring to FIG. 1, a second foot brace support structure 226 having a second foot brace pivot 228 extends from the second foot brace base 216 and attaches to a second foot brace 230 so as to dispose the second foot brace 230 in a position configured to secure a foot of a patient disposed on the operating bed 102. The second foot brace 230 may be identical in structure to the first foot brace 152, discussed above (i.e., may include a plantar support and an achilles support, each including a strap). The second foot brace 230 may be roughly aligned with a second knee brace assembly on the second leg support member 110 in the same manner as described above with respect to the first foot brace 152 and the first knee brace assembly 146.

Referring still to FIG. 1, the second support structure 226 includes a second foot brace pivot 228 configured to allow the second foot brace 230 to pivot relative to the second leg member 110 in order to accommodate different leg sizes and positions. The second foot brace base 216 is removable as desired, such as if the corresponding leg of a patient is not to be distracted. The second foot brace base 216 can be removed from the second track 204 simply by removing the fasteners connecting the second foot brace connector piece 222 and the second foot brace base 216, and sliding the portion or tab of the second foot brace base 216 out of the second foot brace sliding member 214 while holding the second foot brace sliding member 214 still.

Referring to FIGS. 1-3, a second knee brace base 232 can be attached to the second knee brace sliding member 212. As seen best in FIG. 3, the second knee brace base 232 has a portion or tab 140 that slides within a channel 142 of the second knee brace sliding member 212. A second knee brace connector piece 238 attached to the second knee brace base 232 with suitable fasteners, such as screws, so as to form a structure which encircles or surrounds the second leg support member 110 whilst still being able to traverse the second track 204 with movement of the second belt 200 through the second knee brace sliding member's attachment to the second belt 200. Other ways of connecting the second knee brace connector piece 238 to the second knee brace base 232 can include, but are not limited to, a snap fit connection, a nut and bolt connection, or a magnetic connection.

Referring to FIG. 2, a second knee brace assembly 180 may be attached to the second knee brace base 232. The second knee brace assembly 180 may include a third cuff 254 and a fourth cuff 264. The second knee brace assembly 180 may include a second support structure 242 connecting to the second knee brace base 232 through a pivot 244 to allow for relative movement between the second knee brace base 232 and the second support structure 242, and extending to a third branch support 248 which connects to a fourth branch support 250 through a pivot 246 to allow for relative movement between the third branch support 248 and the fourth branch support 250. The pivot 246 can further include an extension feature 160 having a lock mechanism and an extension mechanism configured to extend or retract the distance between the third cuff 254 and the fourth cuff 264. The third branch support 248 connects to the third cuff 254 with a third cuff support 252. The third cuff support 252 may include a plurality of curved arms to which the mesh material of the third cuff 254 may be adhered with a suitable adhesive or other alternative. The fourth branch support 250 is connected to the fourth cuff 264 through a fourth cuff support 262, which may include a plurality of curved arms to which the mesh material of the fourth cuff 264 is adhered with a suitable adhesive.

Referring still to FIG. 2, the third cuff 254 can be one-piece, multiple pieces, one piece attached by hook-and-loop fasteners, or the like. The third cuff 254 is configured to house, restrain, hold, grasp, encircle, or otherwise touch an area of a patient's leg slightly proximal to the knee joint (i.e., a thigh region of the patient's leg). It is advantageous for the third cuff 254 to be one piece because it leaves less room for errors during a procedure. The third cuff 254 has a third inflator 256 and a third pressure gauge 258. The third cuff 254 is configured to be inflated with the third inflator 256, and the third pressure gauge 258 is configured to measure and communicate or display the amount of force exerted on that portion of a patient's lower extremity by the third cuff 254. The third cuff 254 is proximal to a fourth cuff 264 and has a diameter d₃ which is larger than the diameter d₄ of the fourth cuff 264.

The fourth cuff 264 is configured to house, restrain, hold, grasp, encircle, or otherwise touch an area of a patient's leg slightly distal to the knee joint (i.e., a calf region of the patient's leg). In conjunction with the third cuff 254, the fourth cuff 264 allows a surgeon to effectively minimize the forces exerted on the patient's knee during hip distraction. The fourth cuff 264 can be one-piece, multiple pieces, one piece attached by hook-and-loop fasteners, or the like. It is advantageous for the fourth cuff 264 to be one piece because it leaves less room for errors during a procedure. The fourth cuff 264 has a fourth inflator 266 and a fourth pressure gauge 268. The fourth cuff 264 is configured to be inflated with the fourth inflator 266, and the fourth pressure gauge 268 is configured to measure and display or communicate the amount of force exerted on that portion of a patient's knee by the fourth cuff 264.

The third cuff 254 and the fourth cuff 264 each have a mesh-like structure that can be in contact with the patient's skin to prevent slippage of the brace as traction is applied to the patient's leg. Although the third and fourth cuffs 254, 264 are illustrated in this manner, any type of cuffs can be used. Each of the third cuff 254 and the fourth cuff 264 defines an opening which can be substantially parallel with a longitudinal axis of the second leg support member 110, although this can be changed by the pivots 244, 246.

The modular hip distractor 100 utilizes weighted force or manual pulling to be used. The movement of the first brace sliding member 134 distally away from the junction 106 and toward the distal end 112 causes distraction of the patient's hip. The pulling motion is created by turning the associated crank 124, 202 in a first direction, thus performing a hip distraction. When hip distraction is no longer desired, the first and/or second crank 124, 202 is rotated in a second direction, thus allowing the leg to go back into a normal position. The modular hip distractor 100 may be used to apply traction to either or both legs of a patient in a safe and efficient manner.

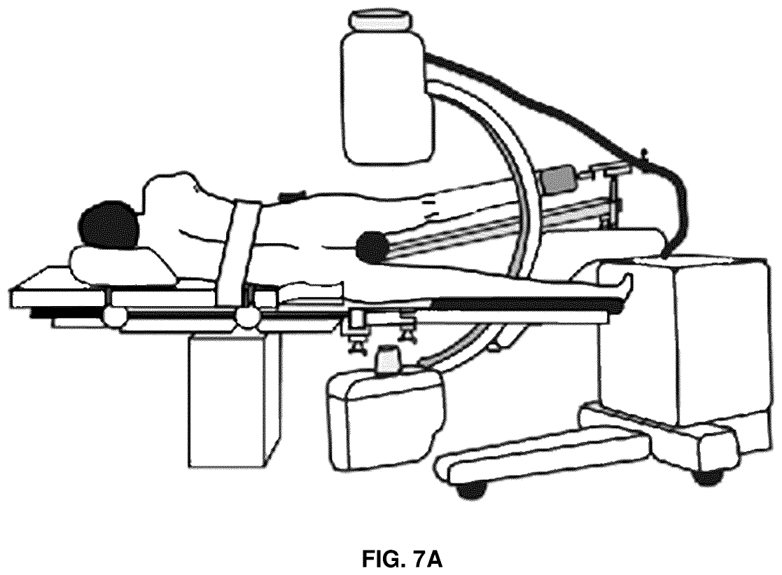

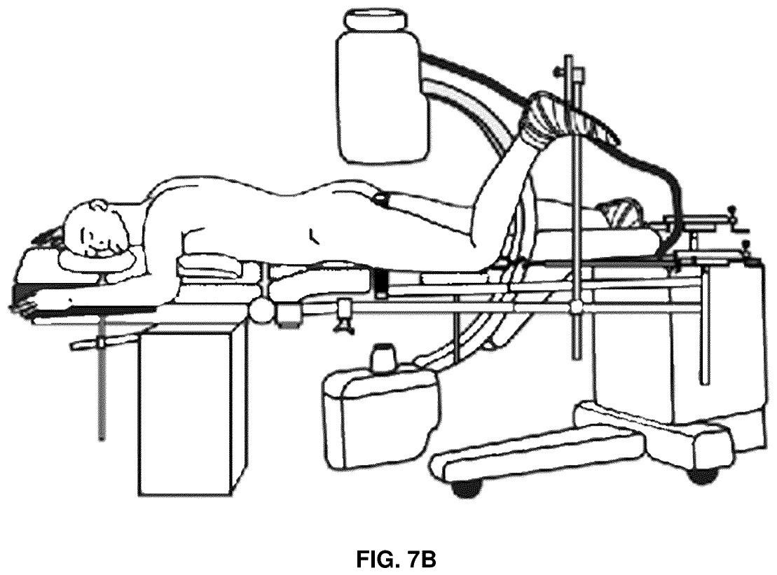

The modular hip distractor 100 offers a mechanical means of hip distraction that does not involve penetration of a patient's soft tissue during use. Referring still to FIGS. 1-4, in operation, the modular hip distractor 100 can be used in an operating room (OR) setting at the operating surgeon's discretion, where both foot braces 152, 230 can be inverted upside-down to accommodate the patient's positioning. For example, a patient can be laid on the operating bed 102 with their legs extending in roughly the same direction as the first and second leg support members 108, 110. The patient will generally be in a relatively horizontal position on the operating bed, typically in supine or Trendelenburg positions, for the duration of their surgery. However, the patient may also be placed in the lateral decubitus (FIG. 7A) or prone (FIG. 7B) position. An optional leg board may be used in case the patient needs to be positioned in the lateral decubitus position. When the patient is in the lateral decubitus or prone position, the first and/or the second foot braces 152, 230 can be adjusted, or inverted upside-down, to accommodate the patient's positioning. The patient's left leg can then be disposed through the first and second cuff 174, 184, and the patient's left foot can be strapped into the first foot brace 152 using the straps 236, 240. The patient's right leg can be disposed through the third and fourth cuffs 254, 264, and the patient's right foot can be strapped into the second foot brace 230 with straps 236, 240. The knee brace assemblies 146, 180 can still be used if the patient is in either the lateral decubitus or prone position.

Air can then be pumped into each of the cuffs 174, 184, 254, 264, if desired, using the corresponding inflaters 176, 186, 256, 266. Both of the patient's legs need not be disposed through the cuffs, as hip distraction may only be desired on one leg. Furthermore, in alternative embodiments, the first cuff 174 and/or the third cuff 254 are the only cuffs used. In other words, each knee brace assembly 146, 180 may only include one cuff instead of two cuffs. When this is the case, the first cuff 174 and/or the third cuff 254 may be used to surround the entire respective knee joint of the patient.

By turning the crank 124, 202 on the side of the leg to be distracted in a direction to cause distal movement of the corresponding knee brace assembly 146, 180 and foot brace 152, 230, the respective leg is pulled on by the corresponding foot brace 152, 230 and knee brace assembly 146, 180 associated with said leg. The modular hip distractor 100 utilizes the knee brace assembly 146, 180 alongside the foot brace 152, 230 to allow for adequate force to be applied to achieve distraction without exerting an undue or unsafe amount of force on the knee joint. When either crank 124, 202 is turned to cause distal movement of the knee brace assembly 146, 180 and the foot brace 152, 230 that is translated into a pulling force on the patient's leg, the force generated is spread out over multiple points of the patient's leg instead of only at the foot. The modular hip distractor 100 generates the pulling force at the foot and ankle as well as in two areas around the knee joint.

Advantageously, the modular hip distractor 100 is adaptable and versatile because of its removable components. The modular hip distractor 100 allows for each of two foot braces 152, 230 and each of two knee brace assemblies 146, 180 to be attached or removed, and moved along the tracks 126, 204.

### Example

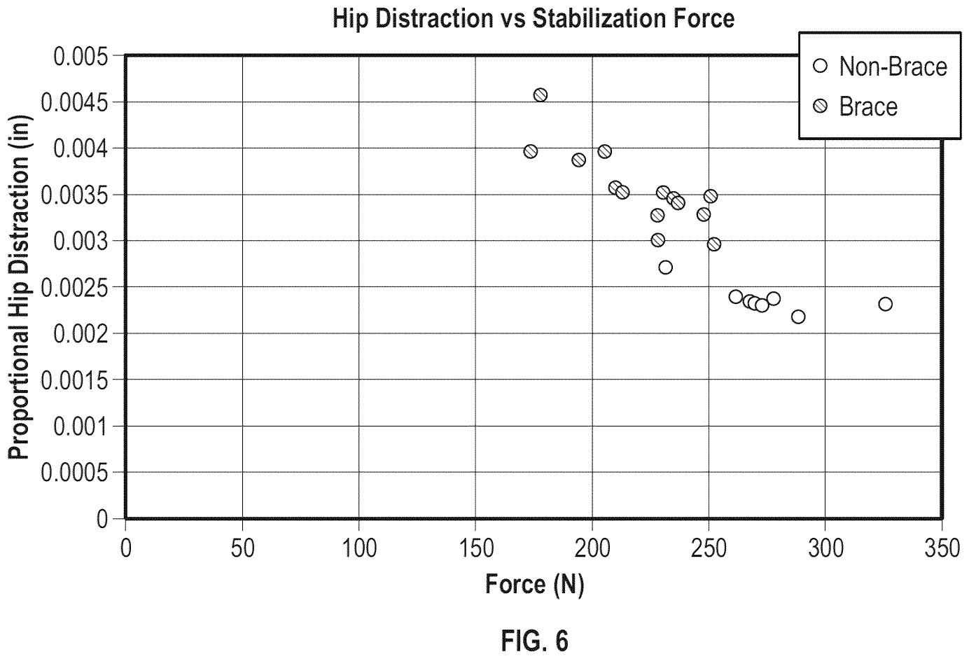

The effects of having a brace around the knee versus having no brace around the knee during a hip distraction were evaluated. A faux leg was placed within a hip distractor with and without a knee brace. A pulling force was then applied to the distal end of the faux leg and readings of force and hip distraction were recorded. The results were based on the comparison between proportional hip distraction and force on the knee. The results, which are shown in FIG. 6, demonstrate that having a brace present around the knee results in a higher hip distraction with a lesser amount of force applied to the lower extremity and knee compared to distraction without a knee brace.

Certain embodiments of the devices and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the devices and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A modular hip distractor comprising:
a first leg support member extending from a first proximal end at a junction to a first distal end;
a second leg support member extending from a second proximal end at the junction to a second distal end;
a first foot brace on the first leg support member;
a first knee brace assembly on the first leg support member between the first proximal end and the first foot brace; and
a crank system configured to move the first foot brace and the first knee brace in concert along the first leg support member;
wherein the junction is configured to extend from, or otherwise attach to, an operating bed.

2. The modular hip distractor of claim 1, wherein the first knee brace assembly comprises a first cuff and a second cuff, wherein the first cuff is disposed along the first leg support member between the junction and the second cuff.

3. The modular hip distractor of claim 2, wherein the first cuff has a first diameter, the second cuff has a second diameter, and the first diameter is larger than the second diameter.

4. The modular hip distractor of claim 2, wherein the first cuff and the second cuff define openings substantially axially parallel with a longitudinal axis of the first leg support member.

5. The modular hip distractor of claim 2, wherein each of the first cuff and the second cuff are mesh-like structures.

6. The modular hip distractor of claim 5, wherein:
the first cuff is attached to a first cuff support comprising a plurality of arms, the mesh-like structure of the first cuff being adhered to the plurality of arms of the first cuff support; and
the second cuff is attached to a second cuff support comprising a plurality of arms, the mesh-like structure of the second cuff being adhered to the plurality of arms of the second cuff support.

7. The modular hip distractor of claim 6, wherein the first cuff support is connected to the second cuff support through a pivot configured to allow relative movement of the first cuff support and the second cuff support.

8. The modular hip distractor of claim 2, wherein the first cuff has a first inflator configured to inflate the first cuff, and/or the second cuff has a second inflator configured to inflate the second cuff.

9. The modular hip distractor of claim 2, further comprising a first pressure gauge configured to measure a pressure exerted on a lower extremity of a patient.

10. The modular hip distractor of claim 1, wherein the first knee brace assembly is movable longitudinally along a track in the first leg support member, and the first foot brace is movable longitudinally along the track.

11. The modular hip distractor of claim 10, wherein the first knee brace assembly and the first foot brace are configured to move upon movement of a belt within the first leg support member.

12. The modular hip distractor of claim 1, further comprising a crank and belt, the belt extending within the first leg support member in an infinite loop between a first pulley near the distal end and a second pulley near the proximal end, wherein the belt is movable in the infinite loop by rotation of the crank.

13. The modular hip distractor of claim 12, further comprising:
a knee brace base connected to a knee brace sliding member disposed on a track on a surface of the first leg support member, wherein the knee brace sliding member is connected to the belt; and
a foot brace base connected to a foot brace sliding member disposed on the track and connected to the belt;
wherein the first knee brace assembly is connected to the knee brace base, and the first foot brace is connected to the foot brace base; and
wherein the knee brace base and the foot brace base are configured to move in concert upon movement of the belt in the infinite loop.

14. The modular hip distractor of claim 13, wherein the knee brace base includes a first tab that fits within a channel of the knee brace sliding member, and the foot brace base includes a second tab that fits within a channel of the foot brace sliding member.

15. The modular hip distractor of claim 13, wherein the knee brace base is attached to a knee brace connector piece so as to surround or encircle the first leg support member, and the foot brace base is attached to a foot brace connector piece so as to surround or encircle the first leg support member.

16. The modular hip distractor of claim 13, wherein the first foot brace is attached to the foot brace base through a foot brace support structure, and the knee brace base is attached to the knee brace through a knee brace support structure.

17. The modular hip distractor of claim 16, wherein the knee brace support structure comprises a first branch support connected to a second branch support through a pivot, wherein the first branch support comprises a plurality of curved arms attached to a first cuff of the knee brace assembly, and wherein the second branch support comprises a plurality of curved arms attached to a second cuff of the knee brace assembly, wherein the first cuff and the second cuff each comprise a mesh-like material.

18. The modular hip distractor of claim 1, wherein the crank system comprises a crank, a first pulley, a second pulley, and a belt running in an infinite loop between the first pulley and the second pulley, wherein the crank is operable to move the belt along the infinite loop.

19. A modular hip distractor comprising first and second leg support members configured to support first and second legs of a patient disposed on an operating bed, wherein at least one of the first and second leg support members includes a knee brace assembly configured to reduce a force exerted on a knee of the first leg of the patient while a hip of the first leg of the patient is distracted in the modular hip distractor.

20. A method of performing a hip distraction, the method comprising:
configuring a patient in a supine, Trendelenburg, lateral decubitus, or prone position;
securing a knee joint of a first leg of the patient with a knee brace assembly, wherein the knee brace assembly comprises a first cuff configured to receive a thigh region of the first leg and a second cuff configured to receive a calf region of the first leg;
securing a foot of the first leg in a foot brace; and
pulling traction on the first leg by pulling a sufficient force simultaneously to the knee brace assembly and the foot brace to cause separation of a femoral head of the first leg from an acetabulum of the first leg.
